(19) United States
(12) Patent Application Publication
(10) Pub. No.: US 2021/0317086 A1
(43) Pub. Date: Oct. 14, 2021

(54) Tramadol HBR-Celecoxib Co-Crystal

(71) Applicant: Mylan Laboratories Limited, Hyderabad (IN)

(72) Inventors: Ramakoteswara Rao Jetti, Hyderabad (IN); Sureshbabu Jayachandra, Hyderabad (IN); Ramamohana Rao Golivi, Hyderabad (IN)

(21) Appl. No.: 17/270,206

(22) PCT Filed: Aug. 16, 2019
(86) PCT No.: PCT/IN2019/050601

§ 371 Date: Feb. 22, 2021

(30) Foreign Application Priority Data
Aug. 20, 2018 (IN) 201841031122

(51) Int. Cl. C07D 231/12; C07C 217/74

(52) U.S. Cl. CPC C07D 231/12; C07C 2601/14; C07B 2200/13; C07C 217/74

(57) ABSTRACT

The present invention provides a novel 1:1 co-crystal of (rac)-tramadol hydrobromide-celecoxib and processes for the preparation of the same by reacting (rac)-tramadol with hydrobromic acid and celecoxib. It also provides crystalline form of (rac)-tramadol hydrobromide.

### CROSS-REFERENCE TO RELATED APPLICATIONS

This application claims the benefit of the earlier filing date of Indian Provisional Patent Application No. IN201841031122 filed on Aug. 20, 2018.

### FIELD OF THE INVENTION

The present invention provides novel co-crystal of (rac)-tramadol hydrobromide and celecoxib.

### BACKGROUND OF THE INVENTION

Tramadol is the compound 2-[(dimethylamino)-methyl]-1-(3-methoxyphenyl) cyclohexanol, widely used as an analgesic in its physiologically acceptable salt forms. (rac)-Tramadol Hydrobromide (HBr) of the present invention has the following structure.

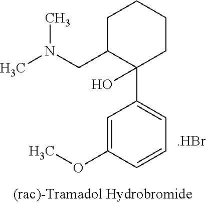 [SMILES: COc1cccc(C2(O)CCCCC2CN(C)C)c1 ; ".HBr"]

Celecoxib is the compound 4-[5-(4-methylphenyl)-3-(trifluoromethyl)-1H-pyrazol-1-yl] benzene sulfonamide has the following structure.

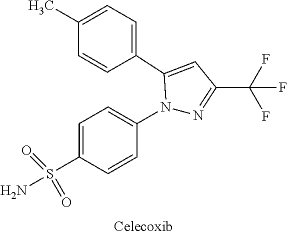 [SMILES: Cc1ccc(-c2cc(C(F)(F)F)nn2-c2ccc(S(N)(=O)=O)cc2)cc1]

A co-crystal comprising (rac)-Tramadol hydrochloride and celecoxib is reported in U.S. Pat. Nos. 8,598,152 and 9,012,440.

### OBJECT AND SUMMARY OF THE INVENTION

The principle object of the present invention is to provide novel 1:1 co-crystal of (rac)-tramadol hydrobromide and celecoxib. It also provides crystalline form of (rac)-tramadol hydrobromide.

In one aspect, present invention provides novel 1:1 co-crystal of (rac)-tramadol hydrobromide and celecoxib.

In another aspect, present invention provides a process for the preparation of 1:1 co-crystal of (rac)-tramadol hydrobromide and celecoxib comprising the steps of:
a) dissolving (rac)-tramadol in an organic solvent;
b) adding hydrobromide;
c) optionally seeding with co-crystal seeds of (rac)-tramadol hydrobromide-celecoxib;
d) adding celecoxib;
e) optionally adding a hydrocarbon solvent; and
f) isolating co-crystal of (rac)-tramadol hydrobromide-celecoxib.

In one more aspect, present invention provides a process for the preparation of 1:1 co-crystal of (rac)-tramadol hydrobromide and celecoxib comprising the steps of:
a) dissolving (rac)-tramadol. HBr and celecoxib in an organic solvent;
b) optionally seeding with co-crystal seeds of (rac)-tramadol hydrobromide-celecoxib;
c) optionally adding a hydrocarbon solvent; and
d) isolating co-crystal of (rac)-tramadol hydrobromide-celecoxib.

In one more aspect, present invention provides crystalline form of (rac)-tramadol hydrobromide.

### BRIEF DESCRIPTION OF THE DRAWINGS

Further aspects of the present disclosure together with additional features contributing thereto and advantages accruing therefrom will be apparent from the following description of embodiments of the disclosure which are shown in the accompanying figures wherein:

FIG. 1: PXRD pattern of (rac)-tramadol. HBr-celecoxib co-crystal (1:1).

FIG. 2. DSC thermogram of (rac)-tramadol.HBr-celecoxib co-crystal (1:1).

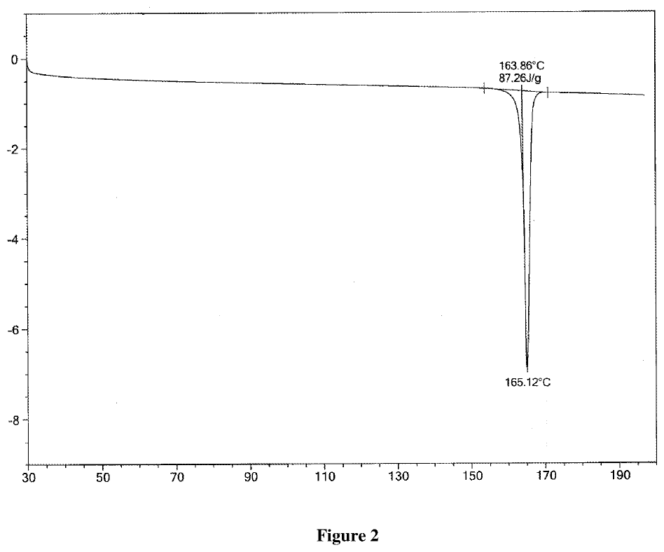

FIG. 3: ¹H NMR spectra of (rac)-tramadol. HBr-celecoxib co-crystal (1:1).

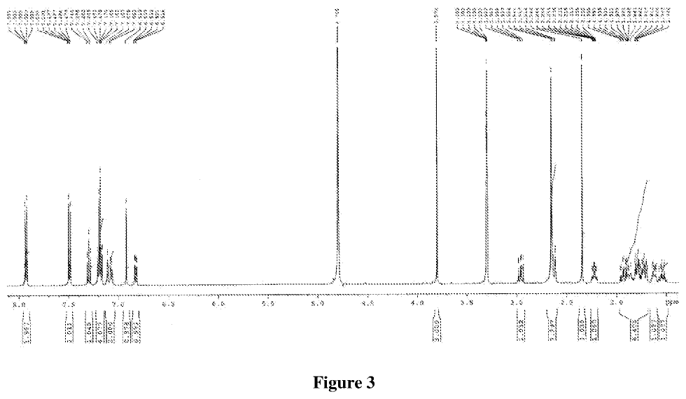

FIG. 4: FT-IR spectra of (rac)-tramadol. HBr-celecoxib co-crystal (1:1).

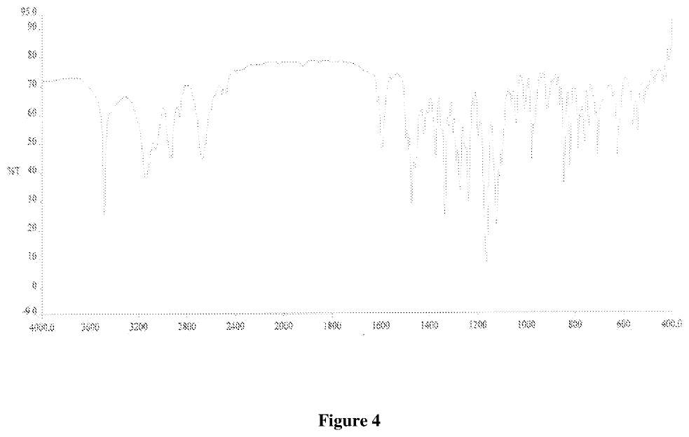

FIG. 5: PXRD pattern of (rac)-tramadol. HBr.

### DETAILED DESCRIPTION OF THE INVENTION

The principle object of the present invention is to provide novel 1:1 co-crystal comprising (rac)-tramadol hydrobromide and celecoxib.

Instrumentation Details:

The PXRD measurements were carried out using PANalytical X′Pert PRO powder diffractometer equipped with goniometer of θ/θ configuration and X'Celerator detector. The Cu-anode X-ray tube was operated at 40 kV and 30 mA. The experiments were conducted over the 20 range of 2.0°-50.0°, 0.030° step size and 50 seconds step time.

Differential Scanning calorimetry (DSC):

The DSC measurements were carried out on TA Q2000/TA Q1000 of TA instruments. The experiments were conducted from 30° C. to 200° C. at a heating rate of 10.0° C./min and nitrogen purging at a flow rate of 50 ml/min. Standard aluminum pans covered by lids with three pin holes were used.

Nuclear Magnetic Resonance (NMR) Spectroscopy:

The ¹HNMR experiments were performed on Bruker 500[RJ1]MHz Avance NMR spectrometer equipped with 5 mm BBO probe in MeOH-d4. The data collected and processed by XWIN-NMR software.

FT-IR:

The FT-IR data was recorded using Perkin Elmer spectrum one instrument in the range of 400-4000 cm¹ by using KBr pellets.

In one embodiment, present invention provides novel 1:1 cocrystal of (rac)-tramadol.HBr-celecoxib.

In another embodiment, present invention provides crystalline 1:1 (rac)-tramadol.HBr-celecoxib co-crystal.

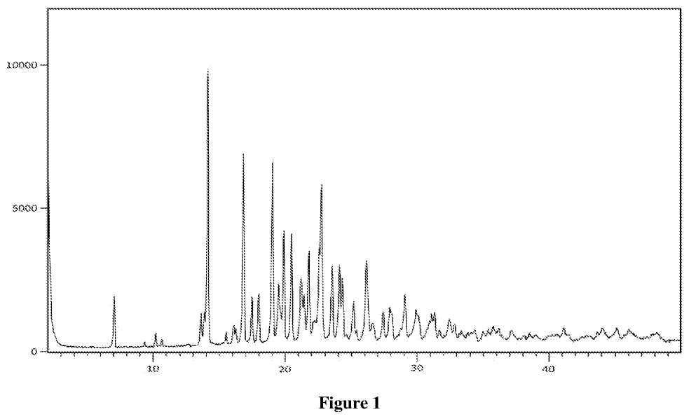

In another embodiment, present invention provides crystalline 1:1 (rac)-tramadol.HBr-celecoxib co-crystal characterized by PXRD pattern substantially as depicted in FIG. 1

Within the context of the present invention, crystalline 1:1 (rac)-tramadol.HBr-celecoxib co-crystal disclosed herein may be characterized by PXRD spectrum having peaks, 14.11, 16.81, 19.02, 19.88, 20.46, 21.77, 22.73, 23.54, 24.09 and 26.16±0.2 0.

In one more embodiment present invention provides a process for the preparation of co-crystal of (rac)-tramadol hydrobromide and celecoxib comprising the steps of:
a) dissolving (rac)-Tramadol in an organic solvent;
b) adding Hydrobromic acid;
c) optionally seeding with 1:1 co-crystal seeds of (rac)-tramadol hydrobromide-celecoxib;
d) adding celecoxib;
e) optionally adding a hydrocarbon solvent; and
f) isolating co-crystal of (rac)-tramadol hydrobromide-celecoxib.

Within the context of the present invention, (rac)-Tramadol is dissolved in an organic solvent includes, but not limited to alcohols, ketones, such as methanol, ethanol, n-propyl alcohol, butyl alcohol, iso-propyl alcohol, acetone, butanone, ethyl isopropyl ketone, methyl isobutyl ketone, methyl ethyl ketone, methyl isopropyl ketone, preferably iso-propyl alcohol. To the solution of (rac)-tramadol is added hydrobromic acid.

In one more embodiment, the hydrobromic acid used in the above step is preferably an aqueous hydrobromic acid solution.

In another embodiment, the above solution is optionally seeded with 1:1 co-crystal seeds of (rac)-tramadol hydrobromide-celecoxib.

In one more embodiment, celecoxib is added to the above reaction solution. The celecoxib used to add in this reaction is preferably dissolved in a suitable solvent includes alcoholic solvents such as methanol, ethanol, n-propyl alcohol, butyl alcohol, iso-propyl alcohol, preferably iso-propyl alcohol.

In one more embodiment, to the above resulting reaction mixture is optionally added hydrocarbon solvent includes but not limited to n-hexane, n-heptane, cyclohexane, cycloheptane, toluene, benzene, ethyl benzene and xylene; preferably n-heptane.

The resulting solid from the above reaction mixture is isolated and washed with suitable solvent to give 1:1 co-crystal of (rac)-tramadol hydrobromide and celecoxib.

In one more aspect, present invention provides a process for the preparation of 1:1 co-crystal of (rac)-tramadol hydrobromide and celecoxib comprising the steps of:
a) dissolving (rac)-tramadol. HBr and celecoxib in an organic solvent;
b) optionally seeding with co-crystal seeds of (rac)-tramadol hydrobromide-celecoxib;
c) optionally adding a hydrocarbon solvent; and
d) isolating co-crystal of (rac)-tramadol hydrobromide-celecoxib.

As per the present invention, (rac)-Tramadol.HBr and celecoxib are dissolved in an organic solvent includes, but not limited to alcohols, ketones, such as methanol, ethanol, n-propyl alcohol, butyl alcohol, iso-propyl alcohol, acetone, butanone, ethyl isopropyl ketone, methyl isobutyl ketone, methyl ethyl ketone, methyl isopropyl ketone, preferably iso-propyl alcohol.

In another embodiment, the above resulting solution is cooled to 50-30° C., more preferably 25-30° C. and optionally seeded with 1:1 co-crystal seeds of (rac)-tramadol hydrobromide-celecoxib.

In one more embodiment, to the above cooled reaction mixture is optionally added hydrocarbon solvent includes but not limited to n-hexane, n-heptane, cyclohexane, cycloheptane, toluene, benzene, ethyl benzene and xylene; preferably n-heptane.

The resulting solid from the above reaction mixture is isolated and washed with suitable solvent to give 1:1 co-crystal of (rac)-tramadol hydrobromide and celecoxib.

Yet another embodiment, present invention provides a crystalline (rac)-tramadol hydrobromide.

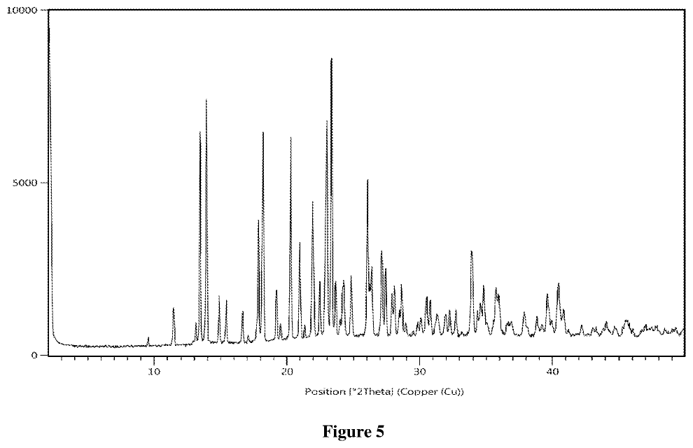

In one embodiment, present invention provides crystalline (rac)-tramadol hydrobromide characterized by PXRD pattern substantially as depicted in FIG. 5.

Within the context of the present invention, crystalline (rac)-tramadol hydrobromide disclosed herein may be characterized by PXRD spectrum having peaks 13.45, 13.93, 17.84 18.19, 20.27, 21.94, 22.98, 23.36, and 26.07±0.2 0.

Indicative Stability:

In yet another embodiment, physical and chemical stability of 1:1 co-crystal of (rac)-tramadol.HBr-celecoxib sample was determined by storing the sample at 40° C./75% relative humidity (RH) and at 25° C./60% relative humidity (RH) for 6 months as mentioned in below Table 1. The samples were analyzed by PXRD and HPLC for final purity.

The 1:1 co-crystal of (rac)-tramadol.HBr-celecoxib shows no change in HPLC purity and PXRD pattern when stored for 6 months at 40° C./75% relative humidity (RH) and at 25° C./60% relative humidity (RH) conditions. This indicates that 1:1 co-crystal of (rac)-tramadol.HBr-celecoxib is physically and chemically stable.

TABLE 1

| | 1:1 Co-crystal of | |
|---|---|---|
| | (rac)tramadol.HBr- | |
| | celecoxib | |
| Condition/ | | HPLC |
| Polymorph | PXRD | purity (%) |

at 40° C./75% RH

| | | |
|---|---|---|
| Initial | Crystalline | 99.89 |
| 1 month | Stable | 99.86 |
| 2 months | Stable | 99.89 |
| 3 months | Stable | 99.88 |
| 6 months | Stable | 99.86 |

at 25° C./60% RH

| | | |
|---|---|---|
| Initial | Crystalline | 99.89 |
| 1 month | Stable | 99.88 |
| 2 months | Stable | 99.89 |
| 3 months | Stable | 99.87 |
| 6 months | Stable | 99.87 |

In view of the above description and the examples below, one of ordinary skill in the art will be able to practice the invention as claimed without undue experimentation. The foregoing will be better understood with reference to the following examples that detail certain procedures for the preparation of molecules according to the present invention. All references made to these examples are for the purposes of illustration. The following examples should not be considered exhaustive, but merely illustrative of only a few of the many aspects and embodiments contemplated by the present disclosure.

### EXAMPLES

Example 1: 1.0 g of (rac)-tramadol was dissolved in 1 mL of IPA at 25±5° C., to this was added 0.64 mL of 48% aqueous solution of HBr and stirred the reaction mass for 3-4 h at 25±5° C. The resulting clear solution was cooled to 10-15° C. and added hot solution of celecoxib (1.44 g of celecoxib dissolved in 5 mL of IPA at 65±5° C.) for 10-15 min at 10-15° C. The reaction mixture was stirred for 1-2 h at 10-15° C. and added 10 mL of heptane; and continued stirring for 1-2 h. The product obtained was filtered, washed with heptane (3 mL) and dried under vacuum. The solid obtained was identified as 1:1 co-crystal of (rac) tramadol HBr-celecoxib.

Yield: 2.27 g

Example 2: The 6.0 g of (rac)-tramadol was dissolved in 6 mL of IPA at 25±5° C. To the clear solution was added 3.85 mL of 48% aqueous solution of HBr and stirred the reaction mass for 3-4 h and cooled to 10-15° C. To the clear solution added seeds (10 mg) of co-crystal of (rac) tramadol.HBr-celecoxib at 10-15° C. Added slowly hot solution of celecoxib (8.69 g) dissolved in IPA (30 ml) at 65±5° C. for 10-15 min at 10-15° C. The reaction mass was stirred for 2-3 h at 10-15° C. and added heptane (60 mL) and continued stirring for 1-2 h. The resulting reaction mass was filtered, washed with heptane (12 mL) and dried under vacuum. The solid obtained was identified as 1:1 co-crystal of (rac) tramadol HBr-celecoxib.

Yield: 13.0 g

Example 3: The 10 g of (rac)-tramadol free base was dissolved in 10 ml of IPA at 25±5° C. and was added 6.4 ml of 48% aqueous solution of HBr and stirred the reaction mass for 3-4 hrs at 25±5° C. The reaction mass was cooled to 10-15° C. To the resulting clear solution was seeded with 10 mg of co-crystal of (rac) tramadol.HBr-celecoxib at 10-15° C. and slowly added hot solution of celecoxib (14.48 g of celecoxib dissolved in 50 ml of IPA) at 65±5° C. for 10-15 min at 10-15° C. and stirred the reaction mass for 2-3 hrs. To the reaction mass was added 100 ml of heptane and continues stirring for further 1-2 hrs. The resulting reaction mass was filtered, washed with heptane and dried under vacuum for 15 h at 25-30° C. The solid obtained was identified as co-crystal of (rac) tramadol HBr-celecoxib.

Yield: 22.0 g

Example 4: The 8.7 g of (rac)-tramadol free base was dissolved in 9 ml of IPA at 25±5° C. and was added 5.6 ml of 48% aqueous solution of HBr at 25±5° C. The reaction mass was stirred for 2-3 h at 25±5° C. and heated to 65-70° C. To the reaction mass was added 18 ml of IPA and continued stirring for 30-40 mins. The reaction mass was cooled to 25±5° C. and further stirred for 1 hr. The reaction mass was filtered, washed with IPA and dried under vacuum at 55° C. for 4 hrs. The solid obtained was identified as (rac)-tramadol hydrobromide salt.

Yield=9.9 g.

Example 5: 9 g of (rac)-tramadol HBr and 10 g of celecoxib were taken in 60 ml of IPA and reaction was heated to 70° C. and stirred for 3-4 hrs. To the reaction mixture 1 ml of water was added. The resulting reaction mixture was cooled to 50° C. and stirred for 4 hrs. Reaction mixture was further cooled to room temperature, was seeded with 10 mg of co-crystal of (rac) tramadol.HBr-celecoxib and stirred for 10 hrs. Reaction mixture was cooled to 10-15° C. then 100 ml of heptane was added and continued stirring for 1-2 hrs. The resulting reaction mass was filtered, washed with heptane and dried under vacuum for 15 h at 25-30° C. The solid obtained was identified as co-crystal of (rac) tramadol HBr-celecoxib.

Yield=17.5 g.

1. A co-crystal comprising (rac)-tramadol hydrobromide and celecoxib.

2. A co-crystal comprising (rac)-tramadol hydrobromide and celecoxib as claimed in claim 1, characterized by a PXRD spectrum having peaks at 14.11, 16.81, 19.02, 19.88, 20.46, 21.77, 22.73, 23.54, 24.09, and 26.16±0.2θ.

3. A co-crystal comprising (rac)-tramadol hydrobromide and celecoxib as claimed in claim 1, characterized by a PXRD pattern substantially as depicted in FIG. 1.

4. A process for the preparation of a co-crystal comprising (rac)-tramadol hydrobromide and celecoxib comprising the steps of:
a) dissolving (rac)-tramadol in an organic solvent;
b) adding hydrobromic acid;
c) optionally seeding with 1:1 co-crystal seeds of (rac)-tramadol hydrobromide-celecoxib;
d) adding celecoxib;
e) optionally adding a hydrocarbon solvent; and
isolating co-crystal of (rac)-tramadol hydrobromide-celecoxib.

5. A process for the preparation of a co-crystal comprising (rac)-tramadol hydrobromide and celecoxib comprising the steps of:
a) dissolving (rac)-tramadol. HBr and celecoxib in an organic solvent;
b) optionally seeding with co-crystal seeds of (rac)-tramadol hydrobromide-celecoxib;
c) optionally adding a hydrocarbon solvent; and
isolating co-crystal of (rac)-tramadol hydrobromide-celecoxib.

6. The process as claimed in claim 4, wherein the co-crystal comprising (rac)-tramadol hydrobromide and celecoxib has PXRD peaks, at 14.11, 16.81, 19.02, 19.88, 20.46, 21.77, 22.73, 23.54, 24.09, and 26.16±0.2θ.

7. The process as claimed in claim 4, wherein the organic solvent is selected from alcohols and ketones.

8. A process as claimed in claim 7, wherein the alcohol solvent is selected from methanol, ethanol, n-propyl alcohol, butyl alcohol, and iso-propyl alcohol and the ketone solvent is selected from acetone, butanone, ethyl isopropyl ketone, methyl isobutyl ketone, methyl ethyl ketone, and methyl isopropyl ketone.

9. The process as claimed in claim 4, where the hydrocarbon solvent is selected from n-hexane, n-heptane, cyclohexane, cycloheptane, toluene, benzene, ethyl benzene and xylene.

10. A crystalline (rac)-tramadol hydrobromide.

11. A crystalline (rac)-tramadol hydrobromide as claimed in claim 10, characterized by a PXRD pattern substantially as depicted in FIG. 5.

12. A crystalline (rac)-tramadol hydrobromide claimed in claim 10, characterized by a PXRD spectrum having peaks at 13.45, 13.93, 17.84, 18.19, 20.27, 21.94, 22.98, 23.36 and 26.07±0.2θ.
